(19) United States
(12) Patent Application Publication
(10) Pub. No.: US 2013/0261208 A1
(43) Pub. Date: Oct. 3, 2013

(54) COMPOSITE HYDROGELS

(71) Applicants: Ana Carolina Borges De Couraca, Sion (CH); Pierre-Etienne Bourban, Nyon (CH); Jan-Anders Edvin Manson, Chexbres (CH); Dominique Pioletti, Buchillon (CH); Arne Vogel, Geneve (CH); Christian Eyholzer, Zurich (CH); Philippe Tingaut, Solothurn (CH); Tanja Zimmermann, Zurich (CH)

(72) Inventors: Ana Carolina Borges De Couraca, Sion (CH); Pierre-Etienne Bourban, Nyon (CH); Jan-Anders Edvin Manson, Chexbres (CH); Dominique Pioletti, Buchillon (CH); Arne Vogel, Geneve (CH); Christian Eyholzer, Zurich (CH); Philippe Tingaut, Solothurn (CH); Tanja Zimmermann, Zurich (CH)

(73) Assignee: ECOLE POLYTECHNIQUE FEDERALE DE LAUSANNE (EPFL), Lausanne (CH)

(21) Appl. No.: 13/808,001

(22) PCT Filed: Jun. 28, 2011
(86) PCT No.: PCT/IB11/52843

§ 371 Date: Jun. 6, 2013

(30) Foreign Application Priority Data
Jul. 1, 2010 (CH) 1078/10

(51) Int. Cl. A61L 31/14

(52) U.S. Cl. CPC A61L 31/145
USPC 522/33; 264/496

(57) ABSTRACT

The present invention relates to reinforced composite hydrogel based on a polymer blend and comprising a network of fibres, said polymer blend comprising UV sensitive molecules. It also relates to a process for preparing the reinforced composite hydrogel according to the invention.

### FIELD OF THE INVENTION

This invention relates to hydrogels. They may advantageously be used in systems were damping and/or transmission of hydrostatic loads is required. Such systems are encountered in engineering devices and in biomedical implants. In biomedical applications for instance it is used for the replacement of tissues such as the nucleus pulposus, the inner core of the intervertebral disc.

### BACKGROUND

Composite Materials

Composite materials have been developed over the last 40 years to meet the need for high performance materials that are stiff, strong and light. These polymer composites can have better specific properties than metals and are widely used in aerospace, automotive, marine, sports industry and biomedical applications [1]. Latest generation of smart composites include active damping or self-healing functions [2, 3]. In the field of biomaterials, composite foams for bone tissue engineering, for example, are composed of PLA and hydroxy apatite, a mineral filler, resulting in materials with high mechanical performance and enhanced bioactivity [4, 5]. Composite foams of PLA with a gradient of fibres content were also developed to tailor the resorption of the scaffolds in vivo. In literature, little data exist on composite hydrogels with tailored mechanical and swelling properties.

Basics of Hydrogels

Hydrogels are defined by Hoffman [6] as hydrophilic polymer networks which may absorb from 10-20% (an arbitrary lower limit) to up to thousands of times their dry weight in water. They can be degradable or not depending on the bonds present in the network. There are three categories of crosslinked gels: entanglements, physical gels, also called reversible gels and chemical or permanent gels.

Gels formed by entanglements are temporary networks, formed when two polymer chains interpenetrate. In physical gels, the networks are held together by secondary forces, including ionic, H-bonding or hydrophobic forces [7, 8]. These gels are not homogeneous, since clusters of molecular entanglements, or hydrophobically- or ionically-associated domains, can create inhomogeneities. Free chain ends and chain loops also represent defects in physical gels.

Hydrogels are called ‘permanent’ or ‘chemical’ gels when they are covalently crosslinked networks. These hydrogels can be generated by crosslinking of water-soluble polymers or by conversion of hydrophobic polymers to hydrophilic polymers, plus crosslinking to form a network [6, 9, 10]. In the crosslinked state, cross-linked hydrogels reach an equilibrium degree of swelling in aqueous solutions, which depends mainly on the crosslink density and on the hydrophilicity of chains. Like physical hydrogels, chemical hydrogels are inhomogeneous because they usually contain regions of low water content and high crosslink density, called clusters. The same defects as in physical gels can be found in chemical gels. These do not contribute to the elasticity of the network in either case. Hydrogels can be classified in a number of other ways. The different macromolecular structures of hydrogels provide one way. These include the following: crosslinked or entangled networks of linear homopolymers, linear copolymers and block or graft copolymers; polyion-multivalent ion, polyion polyion or H-bonded complexes; hydrophilic networks stabilized by hydrophilic domains; and physical blends [6]. Classification can also be based on ionic charges: neutral, anionic, cationic and ampholytic hydrogels; on structure: amorphous, semicrystalline hydrogels. Crosslinking method is another basis for classification. For chemical hydrogels the methods are: crosslinking by radical polymerization, by chemical reaction, by high energy irradiation and using enzymes. For physical hydrogels: crosslinking by ionic interactions, by crystallization and finally physically crosslinked hydrogels from amphiphilic block and graft copolymers [6].

Composite Hydrogels

In the past decades, many efforts have been done to improve the mechanical properties of hydrogels such as introducing dangly comb-chains [11, 12], adding polymer particles in hydrogel networks [13], cold treating [14] or freeze drying [15], incorporating clay with polymer [16-18] and forming interpenetrating network structures (IPN) [11, 19]. Composite hydrogels are composed of at least two components, each with specific functions. Therefore the characteristics of a composite hydrogel depend on the physicochemical properties of the constituents and also on the structure of the material. Indeed, two hydrogels based on the same materials can have different properties by changing the size of the structural elements. The morphology of the structural elements, the nature of interphase interactions, methods of synthesis and ways of combining two phases can also change the final properties of composite hydrogels. The most used polymers for creating a composite hydrogel are hydroxyl-containing polymers (i.e., PVA and its co-polymers, copolymers of 2-hydroxyethyl methacrylate), polyethers (i.e., poly(ethylene oxide), PEO, block copolymers of ethylene oxide and propylene oxide), polymers containing amide groups (i.e., polyacrylamide(PAA), poly(N,N-dimethylacylamide), poly(N-isopropylacrylamide) (PIPA), poly(N-vinylpyrrolidone) (PVP)) [20]. The interactions between these polymers can create block- and graft copolymers as well as IPNs. For the copolymer hydrogels, the presence of ionic and nonionic polar groups on the polymers allows their grouping by various physical and chemical interactions. An example of such complexes is provided by hydrogels based on poly(methacrylic acid) (PMMA) and polyethylene oxide (PEO) [21-25]. The hydrogel is created by formation of hydrogen bonds between carboxyl groups and oxygens of polyether chains. It exhibits a relatively high equilibrium water content in the range of 13-68% while having better mechanical properties than PEO hydrogels alone [21].

The synthesis of IPNs is straightforward because it does not require formation of covalent bonds between two polymers, the bonding of the macromolecules is done by the entanglement of two polymer networks. However other interactions, which will change the properties of the IPN, can take place between the macromolecules such as hydrogen bonding, hydrophobic interactions, ion-dipole interactions and interactions typical of polyelectrolytes systems. There are two ways to synthesize IPNs: in the first case, both networks develop simultaneously and this is the case if networks are formed by two independent mechanisms. In the other case, networks are developed in two stages: the first network appears at the initial stage and when then saturation with components of the second network occurs, the second network forms [20]. This second case allows for better control of the phase separation and morphology of the composite hydrogel. These hydrogels are principally used in biomedical applications that require a certain resistance of the system. An example of such IPNs is provided by hydrogels based on polyacrylate (PAC) and polyamide (PAM) [19, 26].

Hydrogels containing inorganic components are also promising. These inorganic components are introduced in the hydrogels either to modify their properties such as compatibility with biological tissues, mechanical properties and thermo- and pH-responsivity, or to create new properties such as magnetic characteristics and antibacterial properties. There are two ways to prepare these organo-inorganic hydrogels. First, the inorganic additives can be mixed in the form of nano- or microparticles with a solution of water-soluble polymers or monomers followed by their polymerization. The second way consists in the formation of the inorganic phase via the sol-gel process: monomers or a polymer and precursors of inorganic component are added to solution and then inorganic component is transformed into water insoluble particles by various chemical reactions. Finally polymerization of the monomers or crosslinking of the polymer is conducted. The most often used inorganic components are oxides or various clays, water-insoluble salts and metals. Li and al. [27] developed a hydrogel of poly(2-hydroxyethyl methacrylate) (pHEMA) reinforced with titanium dioxide (TiO2) for applications as orthopaedic and dental implants. Haraguchi et al. [17] showed that a hydrogel reinforced with a water-swellable clay could withstand high levels of deformations in torsion and elongation and have a high swelling ratio.

Research on composite hydrogels for biomimetic applications is focused on degradable hydrogels for drug delivery systems, scaffolds for tissue engineering and coatings of biomedical devices. Reinforcement is provided either by active particles, linking the increase of mechanical properties to a biological effect, or by inert particles, where only the reinforcement effect is desired. The choice of fillers for this application is not very broad. Hydroxyapatite and calcium are used for their biological activity whereas clay is used for its bio-inert properties.

The swelling behavior of the hydrogel for this desired application is a key parameter and the ideal filler should have the ability of increasing the mechanical properties without hindering the swelling properties of the hydrogel.

### Fibers for Hydrogel Reinforcement and Tuning of Composite Swelling Behavior

Fibers for mechanical reinforcement of hydrogels covered by this invention can be composed of e.g. natural fibres, silk, collagen, cellulose or polymers. The suitability of a specific fiber for the use in hydrogels can depend on several aspects, as inherent mechanical properties, surface area and dimensions or hydrophilicity. The present invention is based in particular, but not exclusively, on fibers with low diameters and high aspect ratios that also have a hydrophilic nature.

Cellulose consists of glucane chains which are composed of anhydroglucose units that are linearly linked by [beta]-1,4 glycosidic bonds. Depending on the source of cellulose, its degree of polymerization can vary between a few hundred to several thousand monomer units. During biosynthesis the glucane chains combine into segments of parallel alignment (crystalline domains) and fringed regions (amorphous domains) by self assembly. The nanofibers formed by this process have diameters varying from about 2 nm to about 100 nm. Nanofibers with a length of more than 1 μm are denoted “nanofibrils” whereas their shorter parts with a length between 200 and 500 nm and diameters below 10 nm are denoted “nanowhiskers” [28].

### SUMMARY OF INVENTION

The present invention relates to reinforced composite hydrogel based on a polymer blend and comprising a network of fibres, said polymer blend comprising UV sensitive molecules.

The highly swollen hydrogel structures are reinforced with fibres for swelling and mechanical properties control.

The hydrogel precursor solution is composed of UV-sensitive monomers, cured under UV light at a determined intensity and time. The obtained hydrogel properties, in terms of swelling and mechanics, can be tailored by the crosslinker monomer content present in the precursor solution.

The composite hydrogel incorporates at least a portion of fibres, which creates an interpenetrating network with the hydrogel network. The fibres can be modified such that their hydrophilicity can be varied as a function of the amount of chemical moieties added at the fibres surface, thereby permitting control of the swelling capacity and stiffness of the reinforced hydrogel structure. For example fibres based on cellulose or polymers can be considered.

The composite hydrogel is used in systems were damping and/or transmission of hydrostatic loads is required. Such systems are encountered in engineering devices and in biomedical implants. In biomedical applications for instance it is used for the replacement of tissues such as the nucleus pulposus, the inner core of the intervertebral disc.

The present invention also relates to a process for preparing a reinforced composite hydrogel according to the invention.

This process comprises the following steps:
i) Monomers, aqueous solution of photoinitiator and deionised water are mixed manually to obtain a homogeneous precursor solution;
ii) fibres, in their dry form or in the gel form, are added to the precursor solution and stirred with a high-shear mixer during 20 minutes to obtain a good dispersion of the fibres;
iii) the precursor solution with the fibres is then degassed for about 15 minutes under a vacuum of 10 mbar to remove bubbles;
iv) this solution is then casted in cylindrical silicon moulds resistant to UV light and exposed to UV light during 30 minutes;
v) the hydrogel samples are then removed from the moulds and stored in phosphate buffered saline (PBS) to allow swelling equilibrium to be reached; the time needed to reach equilibrium varying between 24 and 48 hours.

### DESCRIPTION OF THE FIGURES

The invention will be better understood below with a detailed description including examples illustrated by the following figures:

FIG. 1: Curing profile of hydrogels as a function of monomer concentration. The monomer is Tween 20 trimethacrylate (T3) and synthesis is described in example 1.

FIG. 2: Micrograph of a non-reinforced hydrogel at swelling equilibrium obtained using a cryo-SEM technique. T3 content 4.5 vol %.

FIG. 3: Stress-strain curves of composite hydrogels at swelling equilibrium as a function of cellulose nanofibrils content.

FIG. 4: Volume increase of composite hydrogel samples at swelling equilibrium. From left to right: hydrogel after polymerization, neat hydrogel, composite hydrogels containing 0.2, 0.4, 0.8 and 1.6 wt % of cellulose nanofibrils.

FIG. 5: Swelling ratio of composite hydrogels

FIG. 6: Micrograph of cellulose nanofibrils reinforced hydrogel at swelling equilibrium obtained using a cryo-SEM technique. T3 concentration 4.5 vol % and cellulose nanofibrils content 0.4 wt %.

FIG. 7: Stress-strain curves of composite hydrogels containing carboxymethylated cellulose nanofibrils with varying DS

FIG. 8: Elastic modulus of composite hydrogels containing carboxymethylated cellulose nanofibrils with varying DS, calculated from the linear part of the stress-strain curves between 20 to 25% of strain.

FIG. 9: Swelling ratio of composite hydrogels containing carboxymethylated cellulose nanofibrils with varying DS.

FIG. 10: Humidity chamber for testing hydrogels in shear

FIG. 11: Micrograph of a composite hydrogel containing carboxymethylated cellulose nanofibrils with a DS of 0.176.

### DETAILED DESCRIPTION

The present invention relates to a composite hydrogel made of a polymer matrix composed of one or more polymers reinforced with nanofibres that create an interpenetrating network. The fibres are disposed in the polymer matrix, creating unique 3-dimensional microstructure and characteristics. The mechanical properties of the reinforced hydrogel, e.g. elastic modulus can be varied as a function of the fibre content thereby permitting control of the stiffness of the structure. The swelling capacity of the hydrogel can also be tuned by the fibre content and the type of fibres used. Thus, the aim consists of producing composite hydrogels that can withstand compressive and hydrostatic loads when hydrated.

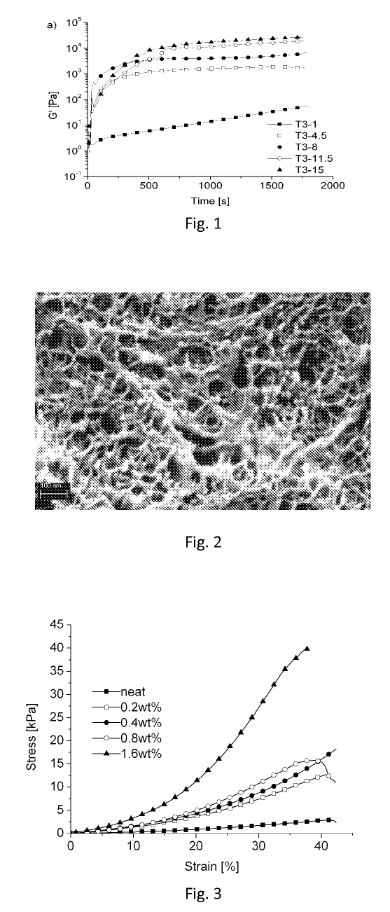

FIG. 1 shows the curing behavior of a hydrogel composed of two monomers sensitive to UV light, photoinitiator and deionised water. The reaction mechanism is described by a radical polymerization. The curing profile determined by photorheology varies with the concentration of branched monomer and the curing time decreases as the branched monomer is increased. The curing profile has three different phases: first, the very steep increase in the storage modulus G′ indicates the creation of new chemical bonds and the formation of the network; secondly, the deceleration step where the curing becomes diffusion-controlled. In this step, after the consumption of the active radicals and the formation of the network, the rate of curing decreases due to the lack of radicals and also to the fact that the remaining radicals are trapped in the newly formed network and cannot diffuse through the latter. Finally, the last step is characterized by a plateau, indication that the reaction is complete. The amount of monomers, photoinitiator and water are generally expressed in volume fraction.

The created hydrogel is a porous structure as observed in FIG. 2. Porosity is defined in terms of relative volume of pores. The pores can be closed or open when the pores are interconnected as it is the case in this invention. An open porosity is important for fluid flow through the structure and for transport of nutriments if living cells are for example introduced in the porous material.

Hydrogels are considered to be weak structures, with a low stiffness of the network. Therefore, to increase the mechanical properties of such hydrated structures, reinforcement is needed. The choice of fillers is of paramount importance. The difference in stiffness of the matrix and the filler should not be important to avoid the creation of stresses at the interfaces. Fibres can be of different aspect ratios between their lengths and diameters and should form a network. The distribution of the fibres can be random or oriented through the structure. The fibres can be used in their dry form or in the form of a gel composed of a certain amount of fibres dispersed in water. The fibres or gel of fibres are mixed with the monomers using a high-shear mixer and are then cured under UV light. The amount of fibres is relative to the amount of polymer matrix and is generally expressed in mass fraction.

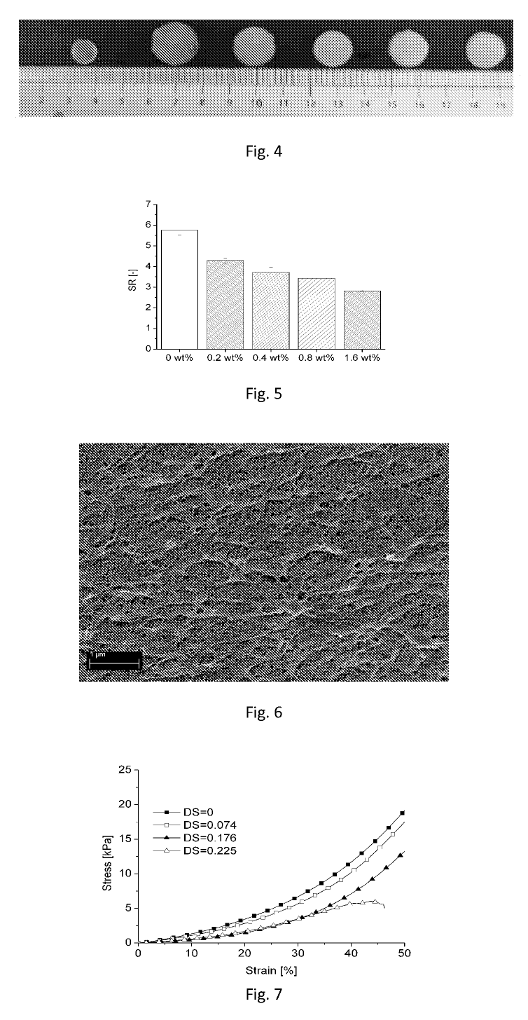

The fibres should also be hydrophilic to insure water uptake of the structure. FIG. 4 shows the volume increase of hydrogel samples at swelling equilibrium with increasing cellulose nanofibril content. The sample on the left is the hydrogel sample after polymerization, i.e. not hydrated. The porosity being defined above as the relative volume of pores, when adding the fibres to the structure, the volume of pores will decrease and subsequently the water absorption will follow the same trend, as observed on FIG. 4. Chemically modified fibres with increased hydrophilicity can be used in order to avoid this limitation.

In the case of a composite hydrogel, as proposed in this invention, mechanical properties and swelling capacity are interdependent. The elastic modulus, i.e. the slope of the linear part of the stress-strain curves of FIG. 3, increases with the cellulose nanofibril content. The swelling capacity, however, decreases with increasing fibril content as observed in FIG. 4. The ideal composite hydrogel designed for a specific application should therefore be a compromise between mechanical performance and swelling ability.

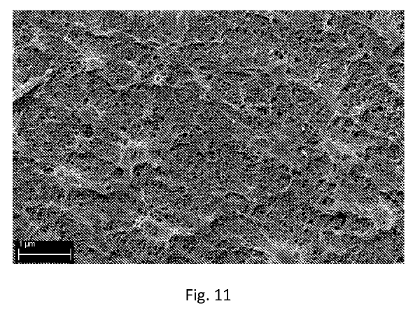

The method of this invention to process the mentioned composite hydrogels is described in detail below. Monomers, aqueous solution of photoinitiator and deionised water are mixed manually to obtain a homogeneous precursor solution. Fibres, in their dry form or in the gel form, are added to the precursor solution and stirred with a high-shear mixer during 20 minutes to obtain a good dispersion of the fibres. The precursor solution with the fibres is then degassed for about 15 minutes under a vacuum of 10 mbar to remove bubbles. This solution is then casted in cylindrical silicon moulds resistant to UV light and exposed to UV light during 30 minutes. The UV intensity can be as high as 145 mW/cm². The hydrogel samples are then removed from the moulds and stored in phosphate buffered saline (PBS) to allow swelling equilibrium to be reached. The time needed to reach equilibrium can vary from 24 to 48 hours. Testing can be performed when the samples are at swelling equilibrium. Special care should be taken with the evaporation of the fluid during testing and adapted set-ups should be developed to obtain reliable measurements. FIG. 10 shows an example of humidity chamber for testing hydrated materials such as hydrogels.

The method can also be used to create composite hydrogels with very specific properties. The matrix can be reinforced by different types of fibres and the degree of shear deformation can be influenced by judicious rearrangement of fibres that could maximize the shear and therefore enhance toughness and impact resistance. This method was used to produce non-degradable composite hydrogels but it could also be used for the production of degradable composite hydrogels given the use of adequate material systems.

Mechanical properties such as elastic modulus as well as swelling capacity can vary on a large range depending on the fibril content and type. Examples will provide values for specific material systems.

### MATERIAL SYSTEMS

In all examples presented in the following sections, the hydrogel matrix was composed of Tween 20® trimethacrylate (T3), n-vinyl-2-pyrrolidone (NVP), photoinitiator Irgacure 2959 as aqueous solution of 0.05 wt % of Irgacure 2959 and deionised water. The T3 concentrations varied from 1 to 15 vol% and the concentrations of NVP from 35 to 49 vol %. The concentration of the Irgacure solution was kept constant at 10 vol % and the amount of water was invariably 40 vol %. Cellulose nanofibrils were used in the upcoming examples. The fibril content varied from 0.2 to 1.6 wt %.

Any molecule that is UV sensitive and polymerizes through a free-radical pathway to produce hydrogels can be used. These include poly(ethylene) dimethacrylate (PEGDMA), hydroxyethyl methacrylate (HEMA) and all acrylic molecules that are able to produce a 3D network.

Concerning the fillers, fibres and mesh of fibres randomly distributed in the matrix or oriented can be used. Fibres are preferably hydrophilic or chemically modifiable to increase their hydrophilicity and have to be deformable with the hydrogel matrix. Some suitable examples can be natural fibres such as silk and flax, wood fibres, cellulose fibres and nanofibres of cellulose and polymer fibres.

### EXAMPLE 1

### Composite Hydrogel Reinforced with Nanofibrils of Cellulose

This example is to illustrate a method for the preparation of a composite hydrogel reinforced with cellulose nanofibrils. In addition, the range of swelling and the mechanical properties are indicated.

Synthesis of T3:

20 g of Tween 20 was dissolved in 100 ml of tetrahydrofuran (THF), to which 6.2 g of 4-(N,N-dimethylamino)pyridine (DMAP) was introduced under argon. After cooling to 0° C., 4.9 ml of methacryloyl chloride (MeOCl) in 30 ml of THF was added dropwise to the mixture over 30 minutes under stirring. The mixture was then protected from light and let stirred overnight at room temperature. The resulting precipitate was then filtered off, washed with THF and dried avoiding exposure to light. The crude product was then purified by column chromatography.

Synthesis of T3/NVP hydrogels reinforced with cellulose nanofibrils (T3 concentration 4.5 vol %): A batch of precursor solution of 6.4 ml was prepared as follows: T3, NVP and photoinitiator were added to a tube. The density of the cellulose nanofibrils gel was assumed to be 1 (gel contains 98% of water). Samples containing 0.2, 0.4, 0.8 and 1.6 wt % of cellulose nanofibrils were prepared by first mixing manually the components and then dispersing the fibrils for 20 minutes using a high shear mixer. The precursor solution was then degassed under vacuum at 10 mbar and finally cast in silicon moulds and UV-cured for 30 minutes at 145 mW/cm².

The surface of the composite hydrogels showed rough regions and clusters, possibly originating from fibrils acting as nucleation points for the matrix (FIG. 6).

The swelling ratio of hydrogels was determined gravimetrically in dependence of time. FIG. 5 shows the swelling ratios at equilibrium for hydrogels with varying cellulose nanofibrils contents. With increasing content of cellulose nanofibrils, the swelling ratio of the composite hydrogels decreased due to stronger crosslinked network.

The mechanical properties in compression of the hydrogels were determined using a universal testing machine. The stiffness of the composite hydrogels was increased with increasing content of cellulose nanofibrils, as shown in FIG. 3.

An increasing content of cellulose nanofibrils therefore increases the stiffness of the composite hydrogel and decreases its swelling ratio at equilibrium. A broad range of properties can therefore be achieved with these composite hydrogels.

### EXAMPLE 2

### Composite Hydrogel Reinforced with Chemically Modified Cellulose Nanofibrils

The objective of the present example is to demonstrate the feasibility of producing composite hydrogels reinforced with chemically modified cellulose nanofibrils and its effect of the swelling and mechanical properties.

Carboxymethylated cellulose nanofibrils with three different degrees of substitution (DS) were prepared: 0.074, 0.176 and 0.225. With increasing DS the hydrophilicity of the carboxymethylated cellulose nanofibrils increases.

The carboxymethylated cellulose nanofibrils were prepared in powder form [29]. The powders were added to the precursor solution and the mixture was homogenized using a high shear mixer. Concentrations of modified fibrils of 0.2, 0.4, 0.8 and 1.6 wt % are used. The hydrogel samples were produced as described in the previous example.

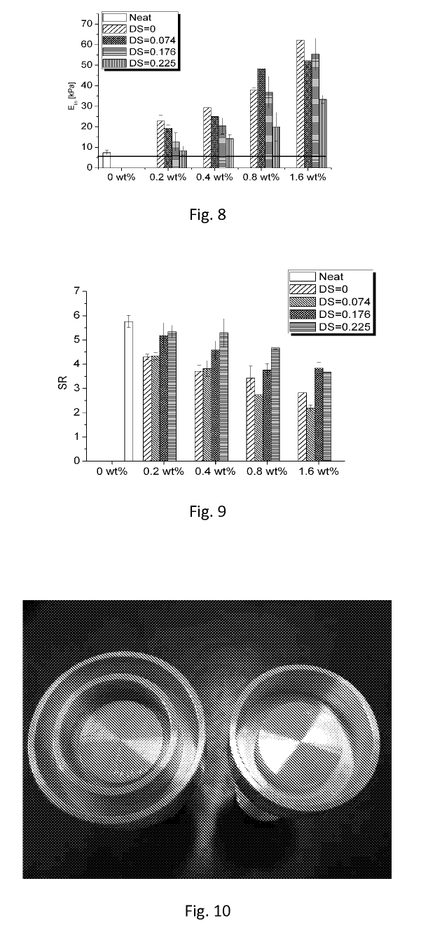

For the same fibril content, the swelling capacity of the composite hydrogels was increased by 1 to 20% with increasing DS due to the hydrophilic functions of the carboxymethylated cellulose nanofibrils (FIG. 9). By increasing the amount of liquid phase in the composite structure, the stiffness of the network was decreased with increasing DS for the same carboxymethylated cellulose nanofibrils contents (FIGS. 7 and 8) but it is still above the results obtained for the non-reinforced hydrogel.

Cryo-SEM micrographs of hydrogels with carboxymethylated cellulose nanofibrils (FIG. 11) showed increased surface roughness. This can be attributed to individual or clusters of carboxymethylated fibrils acting as nucleating points during polymerization.

### REFERENCES

[1] Hull D, Clyne T W. Comprehensive composite Materials. Volume 2: Polymer Matrix Composites.: Elsevier; 1996.
[2] Fischer C, Bennani A, Michaud V, Jacquelin E, Manson J-A, E. Structural damping of model sandwich structures using tailored shear thickening fluid compositions. Smart Materials and Structures. 2010; 19.
[3] Kirkby E L, Michaud V J, Manson J-A E, Sottos N R, White S R. Performance of self-healing epoxy with microencapsulated healing agent and shape memory alloy wires. Polymer. 2009; 50:5533-8.
[4] Mathieu L M, Montjovent M-O, Bourban P-E, Pioletti D P, Manson J-A E. Bioresorbable composites prepared by supercritical fluid foaming. J Biomed Mater Res A. 2005; 75:89-97.
[5] Mathieu L M, Mueller T L, Bourban P-E, Pioletti D P, Muller R, Manson J-A E. Architecture and properties of anisotropic polymer composite scaffolds for bone tissue engineering. Biomaterials. 2006; 27:905-16.
[6] Hoffman A S. Hydrogels for biomedical applications. Advanced Drug Delivery Reviews. 2002; 54(1):3-12.
[7] Campoccia D, Doherty P, Radice M, Brun P, Abatangelo G, Williams D F. Semisynthetic resorbable materials from hyaluronan esterification. Biomaterials. 1998 December; 19(23):2101-27.
[8] Prestwich G D, Marecak D M, Marecek J F, Vercruysse K P, Ziebell M R. Controlled chemical modification of hyaluronic acid: synthesis, applications, and biodegradation of hydrazide derivatives. J Control Release. 1998 Apr. 30; 53(1-3):93-103.
[9] Calvosa G, Bartalesi R, Tenucci M, inventors; INTERSPINOUS VERTEBRAL DISTRACTOR FOR PERCUTANEOUS IMPLANTATION. 2009.
[10] Moehlenbruck J, Chandrashekhar P, inventors; HYDROGEL COMPOSITIONS COMPRISING NUCLEUS PULPOSUS TISSUE. 2004.
[11] Kaneko Y, Nakamura S, Sakai K, Aoyagi T, Kikuchi A, Sakurai Y, et al. Rapid deswelling response of poly(N-isopropylacrylamide) hydrogels by the formation of water release channels using poly(ethylene oxide) graft chains. Macromolecules. 1998 September; 31(18):6099-105.
[12] Yoshida R, Uchida K, Kaneko Y, Sakai K, Kikuchi A, Sakurai Y, et al. Comb-Type Grafted Hydrogels with Rapid De-Swelling Response to Temperature-Changes. Nature. 1995 March; 374(6519):240-2.
[13] Zhang J T, Huang S W, Xue Y N, Zhuo R X. Poly(N-isopropylacrylamide) nanoparticle-incorporated PNIPAAm hydrogels with fast shrinking kinetics. Macromol Rapid Commun. 2005 August; 26(16):1346-50.
[14] Zhang X Z, Zhuo R X. A novel method to prepare a fast responsive, thermosensitive poly(N-isopropylacrylamide)hydrogel. Macromol Rapid Commun. 1999 April; 20(4):229-31.
[15] Kato N, Sakai Y, Shibata S. Wide-range control of deswelling time for thermosensitive poly(N-isopropylacrylamide)gel treated by freeze-drying. Macromolecules. 2003 February; 36(4):961-3.
[16] Haraguchi K, Farnworth R, Ohbayashi A, Takehisa T. Compositional effects on mechanical properties of nanocomposite hydrogels composed of poly(N,N-dimethylacrylamide) and clay. Macromolecules. 2003 July; 36(15):5732-41.
[17] Haraguchi K, Takehisa T. Nanocomposite hydrogels: A unique organic-inorganic network structure with extraordinary mechanical, optical, and swelling/de-swelling properties. Adv Mater. 2002 August; 14(16):1120-4.
[18] Haraguchi K, Takehisa T, Fan S. Effects of clay content on the properties of nanocomposite hydrogels composed of poly(N-isopropylacrylamide) and clay. Macromolecules. 2002 December; 35(27):10162-71.
[19] Hou X P, Siow K S. Novel interpenetrating polymer network electrolytes. Polymer. 2001 April; 42(9):4181-8.
[20] Pavlyuchenko V N, Ivanchey S S. Composite polymer hydrogels. Polym Sci Ser A. 2009 July; 51(7):743-60.
[21] Kim S J, Lee C K, Kim I Y, An K H, Kim S I. Preparation and characterizations of interpenetrating polymer network hydrogels of poly(ethylene oxide) and poly(methyl methacrylate). J Appl Polym Sci. 2003 July; 89(1):258-62.
[22] Osada Y, Sato M. Thermal Equilibrium of Intermacromolecular Complexes of Polycarboxylic Acids Realized by Cooperative Hydrogen-Bonding. Journal of Polymer Science Part C-Polymer Letters. 1976; 14(3):129-34.
[23] Starodubtsev S G, Filippova O Y. Interaction of Polymethacrylic Acid Networks with Polyethylene-Glycol. Vysokomolekulyarnye Soedineniya Seriya B. 1992 July; 34(7):72-9.
[24] Tsuchida E, Abe K. Interactions between Macromolecules in Solution and Intermacromolecular Complexes. Advances in Polymer Science. 1982; 45:1-119.
[25] Yu X H, Tanaka A, Tanaka K, Tanaka T. Phase-Transition of a Poly(Acrylic Acid) Gel Induced by Polymer Complexation. J Chem Phys. 1992 November; 97(10):7805-8.
[26] Kaneko D, Tada T, Kurokawa T, Gong J P, Osada Y. Mechanically strong hydrogels with ultra-low frictional coefficients. Adv Mater. 2005 March; 17(5):535-+.
[27] Li C, Zheng Y F, Lou X. Calcification capacity of porous pHEMA-TiO2 composite hydrogels. J Mater Sci-Mater Med. 2009 November; 20(11):2215-22.
[28] Eichhorn S J, Dufresne A, Aranguren M, Marcovich N E, Capadona J R, Rowan S J, et al. Review: Current international research into cellulose nanofibres and nanocomposites. Journal of Materials Science. 45(1):1-33.
[29] Eyholzer C, Bordeanu N, Lopez-Suevos F, Rentsch D, Zimmermann T, Oksman K. Preparation and characterization of water-redispersible nanofibrillated cellulose in powder form. Cellulose. 2009:1-12.

1. Reinforced composite hydrogel based on a polymer blend and comprising a network of fibres, said polymer blend comprising UV sensitive molecules.

2. Reinforced composite hydrogel according to claim 1 wherein said fibres are modified in a way as to increase their hydrophilicity.

3. Reinforced composite hydrogel according to claim 2 wherein carboxymethyl functions are added at the surface of said fibres.

4. Reinforced composite hydrogel according to claim 1 wherein said fibres are cellulose nanofibres, having diameters between 2 and 100 nm.

5. Reinforced composite hydrogel according to claim 1 wherein the hydrogel matrix is composed of Tween 20® trimethacrylate (T3), n-vinyl-2-pyrrolidone (NVP), photoinitiator Irgacure 2959 as aqueous solution of 0.05 wt % of Irgacure 2959 in water and deionised water, the T3 concentration varying between 1 to 15 vol % and the concentrations of NVP from 35 to 49 vol %, the concentration of the Irgacure solution being kept constant at 10 vol % and the amount of water being invariably 40 vol % and the fibril content varying between 0.2 and 1.6 wt %.

6. Reinforced composite hydrogel according to claim 1 for biomedical applications.

7. Reinforced composite hydrogel according to claim 6 for the replacement of tissues such as the nucleus pulposus.

8. Process for preparing a reinforced composite hydrogel according to claim 1 comprising the following steps:
i) Monomers, aqueous solution of photoinitiator and deionised water are mixed manually to obtain a homogeneous precursor solution;
ii) fibres, in their dry form or in the gel form, are added to the precursor solution and stirred with a high-shear mixer during 20 minutes to obtain a good dispersion of the fibres;
iii) the precursor solution with the fibres is then degassed for about 15 minutes under a vacuum of 10 mbar to remove bubbles;
iv) this solution is then casted in cylindrical silicon moulds resistant to UV light and exposed to UV light during 30 minutes;
v) the hydrogel samples are then removed from the moulds and stored in phosphate buffered saline (PBS) to allow swelling equilibrium to be reached; the time needed to reach equilibrium varying between 24 and 48 hours.
